SEQUENCE LISTING (12 sequences)

SEQ ID NO: 1 - DNA, 801 bp, Homo sapiens
ccacaccaagaaaasaaaaagcaggaactgcatgttgtagatgttcaattttgtcttcatcaccgccgctctttacaaagccatggaatgaaaaacaaaatacacttaaaattgccttcagtttcactatccaagtaaccccaaagaatatccttttasttagggattcgtgttaaagctatggagattacttcctggactstgtgtaggacttgatgattgagagacgatttcktttttatggacccccacccccacccttttcctttctgtttctttctttctttttttctttctttctttctttaacagcaagatgaccaagtaatcagacttctctctgccaggaacgtcccccagcctcatcgtcagcaaacactgttttgcgcacacgtaataacaacaccctggactttaaactggcatagcagctggaaagggaatgggtkggaggggaggggaggaaaggagaggagaggagagatgggtacttcacgccacaaccccctcaaccaagaccacctccgcaaccacagcgggaaaagctaggrgcaggcagcccacttttgcaattactytacattacagtgaactgcggtcctcacgtcttcttttcttcactccccttaacaaaataaaataaataatttcccttcctgcccacccacggttgcccaagaccaagtcttagaggcagccgggttccgatcaagtgctgcgaaacgacgcgtttgacagctgctctggagccgaggascacaataagagaaggcgcgtgaggctccggcagcaaacaccagc

SEQ ID NO: 2 - DNA, 801 bp, Homo sapiens
gcagttaggatggtgaaaaccaatgaaataactgagcattatagtcattatggttgcgaaagttgacaagaaagtaaaacccagkactcccgaatttgtgacacagctttcaaaccataccgtgtactcaaacccttctctctcctgtggctcttattatataatatctatattcaatctcttttctcatathagacagtattactgaagtcttcaytaacatctacgtgaccagttttggcyctgtctcagatacagatatggtgagtttttcattaaatrgtatttttacttcacrtaatggggagtggggggtaatttcaaataaactagcatttttagtgttatagaattatgtatatgtctcatgtctatttagtctactcaaatacatggatagttaargatgtttggatctatggtttccatttccaacttcagtgtttggattagccctatgtattttacgtcattttcaaaacatgtctacctaactacagtgtaataaacaattatttaaataaaattaattgtattaagaagaaacattttctaggaggattaaggcagmattctggacttccataaggaaagtgaatatagtttcacagataaagacttcttacagaattaggtcatggtcagtgtatcacatttaaaaacaacaacaacaacaaaaaaccctytgattggtattgagcaaagtgattgccttgttatgctatctacatacayacaatacgtataatctgcacacatagtaggtatatataaatcaacctaaaaaaattagaatgttgg

SEQ ID NO: 3 - DNA, 801 bp, Homo sapiens
gaaagttgcaatttcccattaaaaaagattatttcctctctgttcctatccatagatrttttttcttggattaattctcaggctattattttctatggctaatgtctcctacccatttctgaccttctagtcttactattttcttggagtttgagagtataaatagacagctactgagaagtagaaaggttggaccagttttctgacttaaagccactgcagtttcaaaatatagctcaggcagttaagaygagatgaaagatttcttttcaccatctctaaggctaatcaggtttattgtttcctgttgttcattatggatgggtatatagttcccaacacaatggaatatagcaatgagtaagtgagtgagtgagtgaataaagttgtcattctgtctcaaactacaaccactccaaatctctccctagctctacaggttcatttatttattattgttttctgtttgccatatatatctactcctatggtagacatgagacacatgtggccatttaaattaaattttaattaaataaaattaaataacatttaaaattttgttccttagtacactggtcatattttatccatatgtggctatgttactatactggatagargagccgaacatttccattatcacagaaagttctgtaaaatagtatttgtctatatcctcccaatagaagttaagttccatgaagactggggttttgtttgctcagaaagataaagccatagcacagaatagcccttgacatagtatatgttcttaataaatcctggttaactaaataa

SEQ ID NO: 4 - DNA, 801 bp, Homo sapiens
acagctgtaaagtggaagtractagtttcacaaacaaataattccraaactactctcttacattcacggatctaggtcacatttcattagagctaaatatatcatgtgtattagcttggataaactatgcacattaccttcttcaaatagaattagtaactttctgtttaccaagtaattttacaaattctcatccaacttgcaaattgaatatatcaagcattcaggatatcctggtggtttctgagatttttaaaacaatttacaacaagacacacataagaattatttgtaagaaaattattttaccttcctttcttgaaattataggtataatttatttttaggttaatcaattcctttctaaaacaaagataaaactctggtaattcaaaycaaatatttcctcactgaaaactatgcttgttaatgaagtataagcggctacatgatcagcagctttacctgtgagacatgtcctttaaatgatattcatagtaatgacaagaataaaaggaaacctgtaatgtcttcattggtataacagcttagattccattctcagtgttttgtattaactcatttaatcttcaaaagtaagaagttattattatttccatttcactgataagaaggtttagacacagaaatattaagccgactcaaggtctcacagtgctggcaaaaggagtagagccctggcaacatggctccagagtttccgtaactactatactatttgtaataacaccttatcatgcgcaattattcagacaaaggcaamaacaataacaacaaaat

SEQ ID NO: 5 - DNA, 23 bp, Artificial Sequence
  Description of Artificial Sequence Synthetic primer
gtcatttcccaacctcaggtctt

SEQ ID NO: 6 - DNA, 29 bp, Artificial Sequence
  Description of Artificial Sequence Synthetic primer
tcacctattacatataaggcttgtgaggt

SEQ ID NO: 7 - DNA, 20 bp, Artificial Sequence
  Description of Artificial Sequence Synthetic probe
ttaatttatttcgcattttt

SEQ ID NO: 8 - DNA, 23 bp, Artificial Sequence
  Description of Artificial Sequence Synthetic probe
tggttaatttatttcacattttt

SEQ ID NO: 9 - DNA, 52 bp, Homo sapiens
aacactgttttgcgcacacgtaataayaacaccctggactttaaactggcat

SEQ ID NO: 10 - DNA, 52 bp, Homo sapiens
tttagtctactcaaatacatggatagwtaaagatgtttggatctatggtttc

SEQ ID NO: 11 - DNA, 52 bp, Homo sapiens
gagtgaataaagttgtcattctgtctyaaactacaaccactccaaatctctc

SEQ ID NO: 12 - DNA, 52 bp, Homo sapiens
ataaaactctggtaattcaaaccaaawatttcctcactgaaaactatgcttg

(19) United States
(12) Patent Application Publication
(10) Pub. No.: US 2015/0057317 A1
(43) Pub. Date: Feb. 26, 2015

(54) GABR-A2 DIAGNOSTIC

(71) Applicant: AstraZeneca AB, Sodertalje (SE)

(72) Inventors: Dennis Joseph MCCARTHY, Wilmington, DE (US); Mark Alan SMITH, Waltham, MA (US)

(73) Assignee: AstraZeneca AB, Sodertalje (SE)

(21) Appl. No.: 14/253,201

(22) Filed: Apr. 15, 2014

Related U.S. Application Data
(63) Continuation of application No. 13/714,841, filed on Dec. 14, 2012, now abandoned.
(60) Provisional application No. 61/570,383, filed on Dec. 14, 2011.

(51) Int. Cl. A61K 31/4402

(52) U.S. Cl. CPC A61K 31/4402
USPC 514/357

(57) ABSTRACT

The present invention provides a method of selection of a patient, who is a candidate for treatment with an NMDA antagonist drug, such as (S)-1-phenyl-2-(pyridin-2-yl)ethanamine or ketamine, whereby to predict an increased or decreased likelihood of response to the NMDA antagonist. The invention provides a method for determining the sequence of GABR-A2 at any of four single nucleotide polymorphism (SNP) sites known as rs3756007, rs11503016, rs17537359 or rs1372472. The method also provides ARMS primers optimised for determining the sequence at these GABR-A2 SNPs and diagnostic kits comprising suitable primers or probes for determining the particular SNPs.

The present invention relates to a method of selection of a patient, who is a candidate for treatment with an NMDA antagonist drug, such as [(S)-1-phenyl-2-(pyridin-2-yl)ethanamine], whereby to predict an increased or decreased likelihood of response to an NMDA antagonist drug and to methods of treating such patients. Part of the invention involves determining the particular polymorphism(s) present at various sites within the nucleic acid sequence of GABR-A2 (the alpha-2 subunit of the GABA receptor). The use of primers, probes and kits capable of detecting these SNPs for predicting likely response to treatment with an NMDA antagonist drug are also part of the invention.

### BACKGROUND/INTRODUCTION

Major Depressive Disorder (MDD) is a psychiatric condition characterized by the presence of one or more depressive episodes without a history of manic, mixed, or hypo-manic episodes. There is evidence for a genetic component in the development of MDD, although a clear pattern of transmission has not been elucidated.

There are currently more than 25 agents approved in the US for the treatment of MDD. Despite the availability of a wide range of antidepressant drugs, clinical trials indicate that 30% to 40% of patients with major depression fail to respond to first-line antidepressant treatment, despite adequate dosage, duration, and compliance. Moreover, there is significant lag time in the onset of antidepressant action with current treatments. Clearly, there is a need to develop novel and improved therapeutic agents for MDD. A therapy for depression with a quicker onset of action and an equal or better tolerability profile than existing therapies would provide a better treatment alternative, one that could potentially reduce both the time that patients suffer with symptoms and the risks associated with suicidal behaviour. (Thase, Journal of Clinical Psychiatry, 63:95-103, 2002).

Studies have demonstrated that the NMDA receptor antagonist ketamine has rapid antidepressant effects in patients with MDD (Berman et al. Biological Psychiatry, 47(4):351-354, 2000 and Zarate et al. Archives of General Psychiatry, 63(8):856-864, 2006).

(S)-1-phenyl-2-(pyridin-2-yl)ethanamine (“COMPOUND A”) is a low-affinity glutamate antagonist (IC₅₀ of 350 nM at the NR1A/2A subtype of the NMDA receptor) which has been shown to be very well tolerated in more than 500 human volunteers and patients with little propensity for causing the psychotomimetic effects comparable to those found with ketamine exposure (Torvaldsson et al. Sleep Research. 14:149-155, 2005; Lees et al. Stroke 322:466-472, 2001; and, Diener et al. Journal of Neurology 249:561-568, 2002). COMPOUND A is disclosed in WO 93/20052, and its use in treating depression is disclosed in WO 00/00540.

The aim of personalised medicine is to predict which treatment offers the best outcome for an individual. Currently it is not possible to gauge likely benefit of an antidepressant for an individual patient.

In the invention described herein, certain single nucleotide polymorphisms (SNPs) in the GABR-A2 gene have been identified which influence how patients with depression or anxiety, such as MDD respond to the NMDA antagonist drug, COMPOUND A. The minor allele of each of SNPs rs3756007, rs1372472, rs11503016 and rs17537359 were found to be associated with an improved outcome in those treated with the NMDA antagonist drug COMPOUND A. SNP rs3756007 was found to possess a particularly strong association. Indeed, this genetic association remains significant even when adjusted for multiple testing is applied (P=0.0336 after adjustment for 16 independent marker loci). Genetic testing of patients with MDD prior to treatment with an NMDA antagonist drug, such as COMPOUND A, at these recited SNP positions (e.g. SNP rs3756007) will assist in identifying those patients, or the patient group, more likely to receive superior benefit from treatment with an NMDA antagonist drug, such as COMPOUND A.

GABA receptors are a family of proteins involved in the GABAergic neurotransmission of the mammalian central nervous system. GABR-A2 is a member of the GABA-A receptor gene family of heteromeric pentameric ligand-gated ion channels through which GABA, the major inhibitory neurotransmitter in the mammalian brain, acts. GABA-A receptors are the site of action of a number of important pharmacologic agents including barbiturates, benzodiazepines, and ethanol. The gene, GABR-A2, is on chromosome 4 and encodes a sub-unit (alpha 2) of the gamma-aminobutyric acid (GABA) receptor.

The minor allele of SNP rs3756007, which shows the association with enhanced response to COMPOUND A is C (cytosine). SEQ ID NO: 1 represents a part of the GABR-A2 gene that contains the rs3756007 SNP. In relation to the sequence shown as SEQ ID NO: 1, the rs3756007 SNP is at position 401 therein.

SEQ ID NO: 2 represents a part of the GABR-A2 gene that contains the rs11503016 SNP. In relation to the sequence shown as SEQ ID NO: 2, the rs11503016 SNP is at position 401 therein.

SEQ ID NO: 3 represents a part of the GABR-A2 gene that contains the rs17537359 SNP. In relation to the sequence shown as SEQ ID NO: 3, the rs17537359 SNP is at position 401 therein.

SEQ ID NO: 4 represents a part of the GABR-A2 gene that contains the rs1372472 SNP. In relation to the sequence shown as SEQ ID NO: 4, the rs1372472 SNP is at position 401 therein.

The sequences in SEQ ID NOs: 1-4 recite the minor allele base at the SNP position.

The various aspects of the invention are particularly useful in connection with the following NMDA antagonist compound: (S)-1-phenyl-2-(pyridin-2-yl)ethanamine (disclosed in WO 93/20052).

The present invention permits the selection of a patient, who is a candidate for treatment with an NMDA antagonist drug, in order to predict an increased or decreased likelihood of response to the NMDA antagonist drug. In particular, if said patient is affected with/suffering from depression and the NMDA antagonist drug is for the treatment of said depression.

According to one aspect of the invention there is provided a method of assessing the suitability of an individual for treatment with an NMDA antagonist, the method comprising, a) in a nucleic acid containing sample taken from the individual, determining the nucleotide at one or more single polynucleotide polymorphic sites selected from: rs3756007, rs11503016, rs17537359, and rs1372472, each of which is a portion of GABR-A2 gene sequence, and assessing the suitability of an individual for treatment with an NMDA antagonist by virtue of the nucleotide present. In one embodiment, the presence of a cytosine at position 401 (according to the SEQ ID NO: 1) or at SNP rs3756007 is indicative of the suitability of the individual to treatment with the NMDA antagonist compound. In one embodiment, the presence of a thymine at position 401 (according to the SEQ ID NO: 2) or at SNP rs11503016 is indicative of the suitability of the individual to treatment with the NMDA antagonist compound. In one embodiment, the presence of a cytosine at position 401 (according to the SEQ ID NO: 3) or at SNP rs17537359 is indicative of the suitability of the individual to treatment with the NMDA antagonist compound. In one embodiment, the presence of a thymine at position 401 (according to the SEQ ID NO: 4) or at SNP rs1372472 is indicative of the suitability of the individual to treatment with the NMDA antagonist compound.

According to one aspect of the invention there is provided a method of assessing the suitability of an individual for treatment with an NMDA antagonist, the method comprising, a) in a nucleic acid containing sample taken from the individual, determining the nucleotide at position 401, according to the position in any one of SEQ ID NOs:1 to 4, each of which is a portion of GABR-A2 gene sequence, and assessing the suitability of an individual for treatment with an NMDA antagonist by virtue of the nucleotide present. In one embodiment, the presence of a cytosine at position 401 (according to the SEQ ID NO: 1) or at SNP rs3756007 is indicative of the suitability of the individual to treatment with the NMDA antagonist compound. In one embodiment, the presence of a thymine at position 401 (according to the SEQ ID NO: 2) or at SNP rs11503016 is indicative of the is suitability of the individual to treatment with the NMDA antagonist compound. In one embodiment, the presence of a cytosine at position 401 (according to the SEQ ID NO: 3) or at SNP rs17537359 is indicative of the suitability of the individual to treatment with the NMDA antagonist compound. In one embodiment, the presence of a thymine at position 401 (according to the SEQ ID NO: 4) or at SNP rs1372472 is indicative of the suitability of the individual to treatment with the NMDA antagonist compound.

According to another aspect of the invention there is provided a method for selecting a patient for treatment with an NMDA antagonist comprising determining the nucleotide at position 401 according to the position in any one of SEQ ID NOs: 1 to 4, each of which is a portion of GABR-A2 gene sequence, in a nucleic acid containing sample obtained from the patient, and selecting the patient for treatment with an NMDA antagonist if the nucleic acid possess the minor allele at said position.

According to another aspect of the invention there is provided a method for selecting a patient for treatment with an NMDA antagonist, the method comprising (i) providing a nucleic acid containing sample from a patient; (ii) determining the nucleotide at position 401, according to the position in any one of SEQ ID NOs:1 to 4, each of which is a portion of GABR-A2 gene sequence, in the nucleic acid of the patient; and selecting the patient for treatment with an NMDA antagonist based thereon. In particular, selecting the patient for treatment with an NMDA antagonist if the nucleic acid of the patient has a cytosine at position 401 (according to SEQ ID NO: 1) and/or thymine at position 401 (according to the SEQ ID NO: 2) and/or a cytosine at position 401 (according to the SEQ ID NO: 3) and/or a thymine at position 401 (according to the SEQ ID NO: 4). I.e., if a patient is homozygous, having a cytosine or thymine at position 401 on both alleles, or is heterozygous, having a cytosine or thymine at position 401 on only one allele.

In a particular embodiment of the aspects of the invention above, when the patient has been determined to be suitable for treatment with an NMDA antagonist, or has been selected for such treatment, the patient is treated with an NMDA antagonist

According to another aspect of the invention there is provided a method of recommending a treatment, the method comprising (a) selecting a patient in need of treatment for depression, the patient's genome having been identified as bearing a cytosine at single nucleotide polymorphism position rs3756007 in GABR-A2 gene, and/or thymine at SNP rs11503016 in GABR-A2 gene, and/or a cytosine at SNP rs17537359 in GABR-A2 gene, and/or a thymine at SNP rs1372472 in GABR-A2 gene; and (b) recommending that the patient be treated with an NMDA antagonist. In one embodiment the patient is particular genotype of the GABR-A2 gene (the particular base present at one or other of the above-mentioned SNPs) is newly diagnosed. In another embodiment the patient or the patient's physician is aware that the patient's GABR-A2 genotype, for example is aware that the patient possesses a cytosine at single nucleotide polymorphism position rs3756007 in GABR-A2 gene from an historical determination.

According to another aspect of the invention there is provided a method of prescribing a treatment for a patient suffering from depression, the method comprising (a) determining whether the patient's genome has been identified as bearing a cytosine at single nucleotide polymorphism position rs3756007 in GABR-A2 gene, and/or thymine at SNP rs11503016 in GABR-A2 gene, and/or a cytosine at SNP rs17537359 in GABR-A2 gene, and/or a thymine at SNP rs1372472 in GABR-A2 gene; and (b) if the patient's genome has been identified as bearing a cytosine at single nucleotide polymorphism position rs3756007 in GABR-A2 gene, and/or thymine at SNP rs11503016 in GABR-A2 gene, and/or a cytosine at SNP rs17537359 in GABR-A2 gene, and/or a thymine at SNP rs1372472 in GABR-A2 gene, prescribing that the patient be treated with an NMDA antagonist.

According to another aspect of the invention there is provided a method of predicting whether or not a patient suffering from depression will benefit from treatment with an NMDA antagonist drug, comprising determining in a nucleic acid containing sample from said patient the nucleotide at SNP position rs3756007, and/or rs11503016, and/or rs17537359, and/or rs1372472, in GABR-A2 gene, wherein presence of a minor allele at any of these SNP positions indicates that the treatment with an NMDA antagonist drug will be more likely to be effective in the individual compared to an individual with the major allele homozygote at said SNP position(s). In one embodiment, the presence of cytosine at SNP position rs3756007 indicates that the treatment with an NMDA antagonist drug will be more likely to be effective in said patient compared to a patient that possesses a thymine at said position.

According to another aspect of the invention there is provided a method for determining the likelihood of effectiveness of an NMDA antagonist drug in treating depression in a patient affected with depression, comprising determining whether the GABR-A2 gene of said patient comprises the minor allele at any one of single nucleotide polymorphism positions rs3756007, rs11503016, rs17537359 or rs1372472, wherein the presence of a minor allele at any of said SNP positions indicates that the NMDA antagonist drug is more likely to be effective in treating depression than if the patients GABR-A2 gene has the major allele homozygote at said positions.

In particular embodiments of the aspects of the invention above, if the GABR-A2 gene possesses a cytosine at rs3756007 the patient is selected for treatment, or is treated, with an NMDA antagonist. In other particular embodiments of the aspects of the invention above, if the GABR-A2 gene possesses a thymine at rs3756007 the patient is not selected for treatment, or is de-selected for treatment, with an NMDA antagonist.

In one embodiment, the methods of the invention comprises determining the sequence of GABR-A2 gene in a sample obtained from the patient at the position corresponding to position 401 as defined in SEQ ID NO: 1. In one embodiment, the method comprises determining whether the sequence of GABR-A2 gene in a sample obtained from the patient at the position corresponding to position 401, as defined in SEQ ID NO:1, is cytosine, whereby to predict an increased likelihood of response to the NMDA antagonist. In one embodiment, the method comprises determining whether the sequence of GABR-A2 gene in a sample obtained from the patient at the position corresponding to position 401 as defined in SEQ ID NO: 1, is thymine.

The methods of the invention are suitable for use with patients with depression and/or anxiety, particularly patients suffering from major depressive disorder (MDD), patients with a single or recurrent depressive episodes, patients with treatment-refractory depression (i.e. patients who have been found to be non-responsive to other depression treatments; TRD), patients with bipolar depression, patients with general anxiety disorder (GAD), patients with obsessive compulsive disorder (OCD), patients with panic disorder, patients with post traumatic stress disorder (PTSD), and patients with social anxiety disorder. Thus, the methods of the invention are suitable for use with patients suffering from: MDD, TRD, GAD, OCD, PTSD, bipolar depression, panic disorder or social anxiety disorder.

There are numerous scientific articles and patent filings describing novel NMDA antagonist compounds. The person skilled in the art is be able to identify an NMDA antagonist for use in the present invention.

Particularly suitable specific NMDA antagonist compounds are selected from: ketamine, and (S)-1-phenyl-2-(pyridin-2-yl)ethanamine (disclosed in WO 93/20052).

The sample obtained from the patient may be any tissue or any biological sample that contains cellular nucleic acid, for example a blood sample containing circulating cells or DNA. In one embodiment the blood sample may be whole blood, plasma, scrum or pelleted blood. In one embodiment a sample is a tissue sample. The tissue sample may be a fresh tissue sample, a frozen sample, a fixed or unfixed sample. In another embodiment the biological sample is a biofluid such as sputum, whole blood—or a blood fraction such as serum or plasma. In another embodiment the biological sample would have been obtained using a minimally invasive technique to obtain the cellular sample, from which to determine one or more of the recited SNP(s) in the GABR-A2 gene sequence. In one embodiment the biological sample must contain sufficient nucleic acid representative of the patient's genome for the identity of the SNP to be detected.

Generation of nucleic acids for analysis from samples generally requires nucleic acid amplification. Many amplification methods rely on enzymatic chain elongation (such as a polymerase chain reaction, ligase chain reaction, or a self-sustained sequence replication). Preferably, the amplification according to the invention is or involves an exponential amplification, such as polymerase chain reaction (PCR).

The particular nucleotide at position 401 (according to any one of SEQ ID NOs: 1 to 4) of the GABR-A2 gene (which correspond to the rs3756007, rs11503016, rs17537359 and rs1372472 SNP, respectively) can be determined by a variety of methods in the art. Particular methods include: polymerase chain reaction (PCR), hybridization with allele specific probes or primers, allele specific amplification (such as amplification refractory mutation system—ARMS), enzymatic mutation detection, mass spectrometry, single strand conformation polymorphisms, restriction fragment length polymorphism (RFLP), WAVE to analysis, denaturing gradient gel electrophoresis, high resolution melting or temperature gradient gel electrophoresis or nucleic acid sequencing.

In one embodiment, the nucleotide at the SNP position in the GABR-A2 gene is determined by sequencing. In another embodiment, the nucleotide at the SNP position in the GABR-A2 gene is determined using a technique that involves polymerase chain reaction (PCR). In a further embodiment, the polymerase chain reaction uses an allele specific primer that detect the base at position 401, as defined in SEQ ID NO: 1, 2, 3 or 4. As noted above, position 401 (according to SEQ ID NO:1) is the SNP known as rs3756007.

In one embodiment of the invention there is provided a method as described hereinabove wherein the method for determining the particular GABR-A2 SNP (rs3756007, rs11503016, rs17537359 or rs1372472)/position 401 of SEQ ID NO: 1-4 in GABR-A2 gene is selected from sequencing, WAVE analysis, restriction fragment length polymorphism (RFLP) and amplification reactions, such as amplification refractory mutation system (ARMS). ARMS is described in European Patent Publication No. 0332435, the contents of which are incorporated herein by reference, which discloses and claims a method for the selective amplification of template sequences which differ by as little as one base, which method is now commonly referred to as ARMS. RFLP is described by Zhong (Zhong et al. 2006 Clinica Chimica Acta: 364, 205-208). In one embodiment of the invention there is provided a method as described hereinabove wherein the method for determining the particular base at SNP rs3756007, rs11503016, rs17537359 or rs1372472, in GABR-A2 gene in a sample obtained from a patient is the amplification refractory mutation system. In one embodiment ARMS may comprise use of an agarose gel, sequencing gel or real-time PCR. In one embodiment ARMS comprises use of real-time PCR. The ARMS assay may be multiplexed with a second PCR reaction that detects the presence of DNA in the reaction, thereby indicating successful PCR. TaqMan™ technology may be used to detect the PCR products of both reactions using TaqMan™ probes labeled with different fluorescent tags. The advantages of using ARMS rather than sequencing or RFLP to detect mutations are that ARMS is a quicker single step assay, less processing and data analysis is required, and ARMS can detect a mutation in a sample against a background of wild type polynucleotide. Amplification reactions are nucleic acid reactions which result in specific amplification of target nucleic acids over non-target nucleic acids.

The polymerase chain reaction (PCR) is a well known amplification reaction.

The term probe refers to single stranded sequence-specific oligonucleotides which have a sequence that is capable of hybridising to the target sequence of the allele to be detected.

The term primer refers to a single stranded DNA oligonucleotide sequence or specific primer capable of acting as a point of initiation for synthesis of a primer extension product which is complementary to the nucleic acid strand to be copied. The length and sequence of the primer must be such that they are able to prime the synthesis of extension products.

The term nucleic acid includes those polynucleotides capable of hybridising, under stringent hybridisation conditions, to the naturally occurring nucleic acids identified above, or the complement thereof. Stringent hybridisation conditions' refers to an overnight incubation at 42° C. in a solution comprising 50% formamide, 5×SSC (750 mM NaCl, 75 mM trisodium citrate), 50 mM sodium phosphate (pH 7.6), 5×Denhardt's solution, 10% dextran sulphate, and 20 pg/ml denatured, sheared salmon sperm DNA, followed by washing the filters in 0.1×SSC at about 65° C., for at least 30 minutes, for example two washes of 30 minutes each.

Many target and signal amplification methods have been described in the literature, for example, general reviews of these methods in Landegren, U., et al., Science 242:229-237 (1988) and Lewis, R., Genetic Engineering News 10:1, 54-55 (1990). These amplification methods can be used in the methods of our invention, and include polymerase chain reaction (PCR), PCR in situ, ligase amplification reaction (LAR), ligase hybridisation, Qβ bacteriophage replicase, transcription-based amplification system (TAS), genomic amplification with transcript sequencing (GAWTS), nucleic acid sequence-based amplification (NASBA) and in situ hybridisation. Primers suitable for use in various amplification techniques can be prepared according to methods known in the art.

Polymerase Chain Reaction (PCR) is a nucleic acid amplification method described inter alia in U.S. Pat. Nos. 4,683,195 and 4,683,202. PCR consists of repeated cycles of DNA polymerase generated primer extension reactions. The target DNA is heat denatured and two oligonucleotides, which bracket the target sequence on opposite strands of the DNA to be amplified, are hybridised. These oligonucleotides become primers for use with DNA polymerase. The DNA is copied by primer extension to make a second copy of both strands. By repeating the cycle of heat denaturation, primer hybridisation and extension, the target DNA can be amplified a million fold or more in about two to four hours. PCR is a molecular biology tool, which must be used in conjunction with a detection technique to determine the results of amplification. An advantage of PCR is that it increases sensitivity by amplifying the amount of target DNA by 1 million to 1 billion fold in approximately 4 hours. PCR can be used to amplify any known nucleic acid in a diagnostic context (Mok et al., (1994), Gynaecologic Oncology, 52: 247-252).

Self-Sustained Sequence Replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al. (1990) Proc. Natl. Acad. Sci. USA 87:1874). Enzymatic degradation of the RNA of the RNA/DNA heteroduplex is used instead of heat denaturation. RNase H and all other enzymes are added to the reaction and all steps occur at the same temperature and without further reagent additions. Following this process, amplifications of 10⁶ to 10⁹ can been achieved in one hour at 42° C.

Ligation amplification reaction or ligation amplification system (LAR/LAS) uses DNA ligase and four oligonucleotides, two per target strand. This technique is described by Wu, D. Y. and Wallace, R. B. (1989) Genomics 4:560. The oligonucleotides hybridise to adjacent sequences on the target DNA and are joined by the ligase. The reaction is heat denatured and the cycle repeated.

Qβ Replicase. In this technique, RNA replicase for the bacteriophage Qβ, which replicates single-stranded RNA, is used to amplify the target DNA, as described by Lizardi et al. (1988) Bio/Technology 6:1197. First, the target DNA is hybridised to a primer including a T7 promoter and a Qβ 5′ sequence region. Using this primer, reverse transcriptase generates a cDNA connecting the primer to its 5′ end in the process. These two steps are similar to the TAS protocol. The resulting heteroduplex is heat denatured. Next, a second primer containing a Qβ 3′ sequence region is used to initiate a second round of cDNA synthesis. This results in a double stranded DNA containing both 5′ and 3′ ends of the Qβ bacteriophage as well as an active T7 RNA polymerase binding site. T7 RNA polymerase then transcribes the double-stranded DNA into new RNA, which mimics the Qβ. After extensive washing to remove any unhybridised probe, the new RNA is eluted from the target and replicated by Qβ replicase. The latter reaction can create a 10⁷ fold amplification in approximately 20 minutes.

Once the nucleic acid has been amplified, a number of techniques are available for detection of single base pair mutations or polymorphisms. One such technique is Single Stranded Conformational Polymorphism (SSCP). SCCP detection is based on the aberrant migration of single stranded mutated DNA compared to reference DNA during electrophoresis. Mutation produces conformational change in single stranded DNA, resulting in mobility shift. Fluorescent SCCP uses fluorescent-labelled primers to aid detection. Reference and mutant DNA are thus amplified using fluorescent labelled primers. The amplified DNA is denatured and snap-cooled to produce single stranded DNA molecules, which are examined by non-denaturing gel electrophoresis.

Chemical mismatch cleavage (CMC) is based on the recognition and cleavage of DNA mismatched base pairs by a combination of hydroxylamine, osmium tetroxide and piperidine. Thus, both reference DNA and mutant DNA are amplified with fluorescent labelled primers. The amplicons are hybridised and then subjected to cleavage using osmium tetroxjde, which binds to an mismatched T base, or hydroxylamine, which binds to mismatched C base, followed by piperidine which cleaves at the site of a modified base. Cleaved fragments are then detected by electrophoresis.

Techniques based on restriction fragment polymorphisms (RFLPs) can also be used.

Furthermore, techniques based on WAVE analysis can be used (Methods Mol. Med. 2004; 108:173-88). This system of DNA fragment analysis can be used to detect single nucleotide polymorphisms and is based on temperature-modulated liquid chromatography and a high-resolution matrix (Genet Test. 1997-98; 1(3):201-6.)

Real-time PCR (also known as Quantitative PCR, Real-time Quantitative PCR, or RTQ-PCR) is a method of simultaneous DNA quantification and amplification (Expert Rev. Mol. Diagn. 2005(2):209-19). DNA is specifically amplified by polymerase chain reaction. After each round of amplification, the DNA is quantified. Common methods of quantification include the use of fluorescent dyes that intercalate with double-strand DNA and modified DNA oligonucleotides (called probes) that fluoresce when hybridised with a complementary DNA.

Specific primers known as Scorpion™ primers can be used for a highly sensitive and rapid DNA amplification system. Such primers combine a probe with a specific target sequence in a single molecule, resulting in a fluorescent detection system with unimolecular kinetics (Nucl. Acids Res. 2000, 28:3752-3761). This has an advantage over other fluorescent probe systems such as Molecular Beacons and TaqMan®, in that no is separate probe is required to bind to the amplified target, making detection both faster and more efficient. A direct comparison of the three detection methods (Nucl. Acids Res 2000, 28:3752-3761) indicates that Scorpions® perform better than intermolecular probing systems, particularly under rapid cycling conditions. The structure of one version of a Scorpion™ primer is such that it is held in a hairpin loop conformation by complementary stem sequences of around six bases which flank a probe sequence specific for the target of interest (Nat. Biotechnol. 1999, 17:804-807). The stem also serves to position together a fluorescent reporter dye (attached to the 5′-end) in close proximity with a quencher molecule. In this conformation, no signal is produced. A PCR-blocker separates the hairpin loop from the primer sequence, which forms the 3′-end of the Scorpion®. The blocker prevents read-through, which would lead to unfolding of the hairpin loop in the absence of a specific target. During PCR, extension occurs as usual from the primer. After the subsequent denaturation and annealing steps, the hairpin loop unfolds and, if the correct product has been amplified, the probe sequence binds to the specific target sequence downstream of the primer on the newly synthesised strand. This new structure is thermodynamically more stable than the original hairpin loop. A fluorescent signal is now generated, since the fluorescent dye is no longer in close proximity to the quencher. The fluorescent signal is directly proportional to the amount of target DNA.

An alternative Scorpion™ primer comprises a duplex of two complementary labelled oligonucleotides. One oligonucleotide of the duplex is labelled with a 5′ end reporter dye and carries both the blocker non-coding nucleotide and PCR primer elements, while the other oligonucleotide is labelled with a 3′ end quencher dye. The mechanism of action is then essentially the same as the Scorpion™ hairpin primer described above: during real-time quantitative PCR, the 5′ end reporter and 3′ end quencher dyes are separated from each other leading to a significant increase in fluorescence emission.

Scorpions™ can be used in combination with the Amplification Refractory Mutation System (ARMS) (Nucl. Acids Res. 1989, 17:2503-2516, Nat. Biotechnol. 1999, 17:804-807) to enable single base mutations to be detected. Under the appropriate PCR conditions a single base mismatch located at the 3′-end of the primer is sufficient for preferential amplification of the perfectly matched allele (Newton et al., Nucl. Acids Res. 17:2503-2516, 1989), allowing the discrimination of closely related species. The basis of an amplification system using the primers described above is that oligonucleotides with a mismatched 3′-residue will not function as primers in the PCR under appropriate conditions. This amplification system allows genotyping solely by inspection of reaction mixtures after agarose gel electrophoresis. It is simple and reliable and will clearly distinguish heterozygotes at a locus from homozygotes for either allele. ARMS does not require restriction enzyme digestion, allele-specific oligonucleotides as conventionally applied, or the sequence analysis of PCR products.

In one embodiment, the nucleotide determination method involves use of real time polymerase chain reaction (real time-PCR) with allele specific (ARMS) primers that detect single base mutations or polymorphisms.

With respect to the base/nucleotide at at rs3756007/position 401 of GABR-A2 gene (according to position in SEQ ID NO: 1), a cytosine is referred to herein as minor allele and thymine is referred to as the major allele. Surprisingly it has been found that patients suffering from depression that posses the minor allele are more likely to resond favourably to treatment for depression with an NMDA antagonist than those that possess the major alleles (homozygote) at rs3756007 in GABR-A2 gene.

In one embodiment of the invention there is provided an ARMS method as described hereinabove wherein a first primer pair is used to detect the minor allele and a second primer pair is used to detect the major allele; and wherein one primer of each pair comprises:—

(a) a primer with a terminal 3′ nucleotide that is specific for a particular allele; and
(b) possible additional mismatches at the 3′ end of the primer.

In one scenario, one primer of each pair comprises:—

(a) a single molecule or nucleic acid duplex probe containing both a primer sequence and a further sequence specific for the target sequence;
(b) a fluorescent reporter dye attached to the 5′ end of the probe in close proximity with a quencher molecule within said single molecule or nucleic acid duplex;
(c) one or more non-coding nucleotide residues at one end of said probe;
(d) wherein said reporter dye and quencher molecule become separated during amplification of the target sequence.

In one embodiment, the probe is a Scorpion™ probe.

In one embodiment of the invention there is provided a method of determining the sequence of GABR-A2 gene at SNP rs3756007, rs11503016, rs17537359 or rs1372472 in a nucleic acid containing sample obtained from a patient comprising use of an ARMS primer capable of recognising the sequence of GABR-A2 gene corresponding to position 401 according to SEQ ID NO: 1-4, respectively. In one embodiment of the invention there is provided a method of determining the sequence of GABR-A2 gene at SNP rs3756007 in a sample obtained from a patient comprising use of an ARMS primer and a companion primer optimized to amplify the region of a GABR-A2 gene sequence comprising the base corresponding to position 401 according to SEQ ID NO: 1. The skilled person would understand that “optimized to amplify” comprises determining the most appropriate length and position of the forward primer and reverse primer. In one embodiment the ARMS primer capable of recognising the particular SNP base can be either of the forward or reverse primers. The forward reverse primers for use in the ARMS assay are optimized to amplify a region of less than 500 bases. In one embodiment the primers are optimized to amplify a region of less than 250 bases. In one embodiment the primers are optimized to amplify a region of less than 200 bases. In one embodiment the primers are optimized to amplify a region of greater than 100 bases.

In one embodiment the ARMS forward primer is capable of recognising the sequence of GABR-A2 gene at the position corresponding to position 401 as defined in any of SEQ ID NOs: 1, 2, 3 or 4. In one embodiment the ARMS reverse primer is capable of recognising the sequence of GABR-A2 gene at the position corresponding to position 401 as defined in any of SEQ ID NOs: 1, 2, 3 or 4. “Recognising” in this context, means specifically hybridising to and/or capable of facilitating primer extension therefrom. Either of the primers used in the ARMS assay may include locked nucleic acids to enhance or facilitate hybridisation to the substrate nucleic acid. Locked Nucleic Acid (LNA) oligonucleotides contain a methylene bridge connecting the 2′-oxygen of ribose with the 4′-carbon. This bridge results in a locked 3′-endo conformation, reducing the conformational flexibility of the ribose, and increasing the local organisation of the phosphate backbone. Braasch and Corey have reviewed the properties of LNA/DNA hybrids (Braasch and Corey, 2001, Chemistry & Biology 8:1-7).

Several studies have shown that primers comprising LNAs have improved affinities for complementary DNA sequences. Incorporation of a single LNA base can allow melting temperatures (Tm) to be raised by up to 41° C. when compared to DNA:DNA complexes of the same length and sequence, and can also raise the Tm values by as much as 9.6° C. Braasch and Corey propose that inclusion of LNA bases will have the greatest effect on oligonucleotides shorter than 10 bases.

Implications of the use of LNA for the design of PCR primers have been reviewed (Latorra, Arar and Hurley, 2003, Molecular and Cellular Probes 17, 253-259). It was noted that firm primer design rules had not been established but that optimisation of LNA substitution in PCR primers was complex and depended on number, position and sequence context. Ugozolli et al (Ugozolli, Latorra, Pucket, Arar and Hamby, 2004, Analytical Biochemistry 324, 143-152) described the use of LNA probes to detect SNPs in real-time PCR using the 5′ nuclease assay. Latorra et al (Latorra, Campbell, Wolter and Hurley, 2003, Human Mutation 22, 79-85) synthesised a series of primers containing LNA bases at the 3′ terminus and at positions adjacent to the 3′ terminus for use as allele specific primers. Although priming from mismatched LNA sequences was reduced relative to DNA primers, optimisation of individual reactions was required.

In one embodiment the ARMS forward and/or reverse primer comprises a sequence in which one or more of the standard DNA bases have been substituted with a LNA base.

In one embodiment there is provided an ARMS probe capable of binding to the amplification product resulting from use of a pair of ARMS primer as described hereinabove in an ARMS assay. In one embodiment the ARMS probe comprises a sequence in which one or more of the standard DNA bases have been substituted with a LNA base. In one embodiment the ARMS probe comprises a Yakima Yellow™ fluorescent tag on the 5′ end. In one embodiment the ARMS probe comprises a BHQ™ quencher on the 3′ end. The skilled person would recognise that the position at which the probe binds in the amplified product (and thus the sequence of the probe is complementary to) is restricted only by the boundaries imposed by the forward and reverse primers which determine the amplified product.

The Control probe can be used to confirm that the ARMS assay is working as intended and to confirm that there is DNA in the sample used in the ARMS assay. The skilled person would understand that the Control probe could be targeted to any chosen gene.

In another aspect of the invention there is provided a method as described hereinabove wherein the method for determining the sequence of GABR-A2 gene at SNP rs3756007 comprises determining the sequence of cDNA generated by reverse transcription of GABR-A2 gene mRNA extracted from the patients' biological sample. Such sample may be a fresh sample, archival sample or other clinical material. Extraction of RNA from formalin fixed tissue has been described in Bock et al., 2001 Analytical Biochemistry: 295 116-117, procedures for extraction of RNA from non-fixed tissues, and protocols for generation of cDNA by reverse transcription, PCR amplification and sequencing are described in Sambrook, J. and Russell, D. W., Molecular Cloning: A Laboratory Manual, the third edition, Cold Spring Harbor Laboratory Press, Cold Spring Harbor, N.Y., 2001.

A further aspect of the invention provides a diagnostic kit comprising a hybridisation or amplification primer capable of identifying the minor allele and a hybridisation or amplification primer capable of identifying the major allele of one of the GABR-A2 SNPs selected from the group consisting of: rs3756007, rs11503016, rs17537359 and rs1372472, and optionally instructions for use.

A further aspect of the invention provides a diagnostic kit, comprising an ARMS forward or reverse primer capable of detecting the rs3756007 SNP in GABR-A2 (corresponding to position 401, as defined in SEQ ID NO: 1), and optionally an ARMS companion primer, and optionally instructions for use. Depending on which primer (forward or reverse) binds at the location of the allele to be detected, the other primer is referred to as the “companion” primer. In one embodiment of the invention there is provided a diagnostic kit, comprising an ARMS mutant primer comprising one or more LNA bases and capable of recognising the sequence of GABR-A2 at the position corresponding to position 401 as defined in SEQ ID NO: 1, and optionally an ARMS companion primer, and optionally instructions for use. In one embodiment the diagnostic kit may be used in a method of predicting the likelihood that a patient, who is a candidate for treatment with an NMDA antagonist, will respond to said treatment. In an alternative embodiment the diagnostic kit may be used in selecting a patient, who is a candidate for treatment for depression with an NMDA antagonist, for said treatment.

In a further aspect of the invention there is provided the use of a primer or a probe capable of recognising thymine or cytosine at the position corresponding to position 401 according to SEQ ID NO: 1, for predicting the response of a patient to treatment for depression with an NMDA antagonist.

In a further aspect of the invention there is provided the use of a primer or a probe capable of recognising thymine or adenine at the position corresponding to position 401 according to SEQ ID NO: 2, for predicting the response of a patient to treatment for depression with an NMDA antagonist.

In a further aspect of the invention there is provided the use of a primer or a probe capable of recognising thymine or cytosine at the position corresponding to position 401 according to SEQ ID NO: 3, for predicting the response of a patient to treatment for depression with an NMDA antagonist.

In a further aspect of the invention there is provided the use of a primer or a probe capable of recognising thymine or adenine at the position corresponding to position 401 according to SEQ ID NO: 4, for predicting the response of a patient to treatment for depression with an NMDA antagonist.

In a further aspect there is provided the use of a primer or probe specific for position 401 of GABR-A2 gene according to SEQ ID NO: 1, or position 401 of GABR-A2 gene according to SEQ ID NO: 2, or position 401 of GABR-A2 gene according to SEQ ID NO: 3, or position 401 of GABR-A2 gene according to SEQ ID NO: 4, in the manufacture of a composition or kit for predicting the response of a patient suffering from depression to an NMDA antagonist.

In a further aspect of the invention there is provided an oligonucleotide at least 12 nucleobases in length, such as at least 12, 15, 20, 25, 30, 35, 40, 45, 50, 75, 100 or more, identical or partly complementary to a sequence that includes the base at the position corresponding to position 401 according to any of SEQ ID NOs: 1-4. In a particular embodiment the oligonucleotide is less than 50 nucleobases. When the sequence is partly complementary to the target sequence, it must be capable of hybridising to said sequence so as to allow detection and/or strand elongation therefrom.

In a specific embodiment, the methods as described hereinabove may be used to assess the pharmacogenetics of an NMDA antagonist. Pharmacogenetics is the study of genetic variation that gives rise to differing response to drugs. By determining the sequence of GABR-A2 gene at SNP rs3756007, or any of the other 3 recited GABR-A2 gene SNPs, in a sample obtained from a patient and analysing the response of the patient to an NMDA antagonist, the pharmacogenetics of the NMDA antagonist can be elucidated.

In one embodiment the method for predicting the likelihood that a patient who is a candidate for treatment with an NMDA antagonist will respond to said treatment, may be used to select a patient, or patient population, with depression for treatment with an NMDA antagonist.

In one embodiment the method for predicting the likelihood that a patient suffering from depression or anxiety who is a candidate for treatment with an NMDA antagonist will respond to said treatment, may be used to predict the responsiveness of a patient, or patient population, with depression to treatment with an NMDA antagonist.

The NMDA antagonist will be incorporated into a composition or formulation suitable for pharmaceutical administration to a subject in need thereof, by, for example, mixing the compound with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, N.Y., USA); Remington's Pharmaceutical Sciences, 20th edition, pub. Lippincott, Williams & Wilkins, 2000 or Handbook of Pharmaceutical Excipients, 2nd edition, 1994.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, is boluses, electuaries, or aerosols.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g. compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichoro-tetrafluoroethane, carbon is dioxide, or other suitable gases.

Formulations suitable for rectal administration may be presented as a suppository with a suitable base comprising, for example, cocoa butter or a salicylate

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

The size of the dose of each therapy which is required for the therapeutic or prophylactic treatment of a particular disease state will necessarily be varied depending on the host treated, the route of administration and the severity of the illness being treated.

Accordingly the optimum dosage may be determined by the practitioner who is treating any particular patient, and taking into consideration various factors known to modify the action of drugs including severity and type of disease, body weight, sex, diet, time and route of administration, other medications and other relevant clinical factors. It may also be necessary or desirable to reduce the above-mentioned doses of the components of the combination treatments in order to reduce toxicity. Therapeutically effective dosages may be determined by either in vitro or in vivo methods.

The compositions described herein may be in a form suitable for oral administration, for example as a tablet or capsule, for nasal administration or administration by inhalation, for example as a powder or solution, for parenteral injection (including intravenous, subcutaneous, intramuscular, intravascular or infusion) for example as a sterile solution, suspension or emulsion, for topical administration for example as an ointment or cream, for rectal administration for example as a suppository or the route of administration may be by direct injection into the tumour or by regional delivery or by local delivery.

Therapeutically effective dosages may be determined by either in vitro or in vivo methods. Calculating therapeutic drug dose is a complex task requiring consideration of medicine, pharmacokinetics and pharmacogenetics. The therapeutic drug dose for a given patient will be determined by the attending physician, taking into consideration various factors known to modify the action of drugs including severity and type of disease, body weight, sex, diet, time and route of administration, other medications and other relevant clinical factors. The NMDA antagonist will however normally be administered to a warm-blooded animal so that a daily dose in the range, for example, 0.01 mg/kg to 75 mg/kg body weight is received, given, if required, in divided doses. The NMDA antagonist may be administered orally such as in a tablet, cachet or capsule. The NMDA antagonist may also be administered parenterally. In such cases lower doses will be used. Thus, for example, for intravenous administration, a dose in the range 0.01 mg/kg to 30 mg/kg body weight will generally be used.

In one embodiment the NMDA antagonist is selected from the group consisting of: (S)-1-phenyl-2-(pyridin-2-yl)ethanamine and ketamine.

An effective amount of an NMDA antagonist will depend, for example, upon the therapeutic objectives, the route of administration, and the condition of the patient. Accordingly, it is possible for the therapist to titer the dosage and modify the route of administration as required to obtain the optimal therapeutic effect. A typical daily or intermittent dosage, such as weekly, fortnightly or monthly, might range from about 0.5 mg to up to 300 mg, 500 mg, 1000 mg or 1200 mg or more, depending on the factors mentioned above.

We contemplate that an NMDA antagonist may be used as monotherapy or in combination with other drugs. The present invention is also useful in adjuvant, or as a first-line therapy.

In one embodiment the methods of the present invention additionally comprises administration of an NMDA antagonist to a patient selected for, or predicted to respond to treatment with an NMDA antagonist according the methods described hereinabove.

In one embodiment the methods carried out on a patient's biological sample to determine the allele at the position in GABR-A2 gene corresponding to position 401 (according to one or other of SEQ ID NOs: 1-4), further comprise administering an amount of an NMDA antagonist to the patient identified as suitable for treatment with the drug. In a particular embodiment the NMDA antagonist is administered to the patient after the determination step.

In a further aspect of the invention there is provided use of an NMDA antagonist in preparation of a medicament for treating a patient, or a patient population, selected for, or predicted to respond to or more favourably to, treatment with an NMDA antagonist according the methods described hereinabove.

In a further aspect of the invention there is provided a method of treating a patient, or a patient population, selected for, or predicted to have an increased likelihood of response to an NMDA antagonist according to the method as described herein, comprising administering an NMDA antagonist to said patient(s).

In a further aspect of the invention there is provided a method of treating a patient suffering from depression or anxiety comprising determining whether or not the patient will respond favourably to an NMDA antagonist according the methods of the invention described above, and administering an effective amount of an NMDA antagonist to said patient if they are identified as likely to be responsive to treatment with an NMDA antagonist.

In a further aspect of the invention there is provided a method of treating a patient suffering from depression or anxiety comprising:
(i) providing a nucleic acid containing sample from a patient
(ii) determining the allele at one or other of the SNPs rs3756007, rs11503016, rs17537359 or rs1372472 in GABR-A2 gene; and
(iii) administering to the patient an effective amount of an NMDA antagonist if the patient's cellular DNA possesses the minor allele at said position.

In a further aspect of the invention there is provided a method of treating a patient suffering from depression or anxiety comprising:
(iv) providing a nucleic acid containing sample from a patient
(v) determining the allele at SNP position rs3756007 in GABR-A2 gene; and
(vi) administering to the patient an effective amount of an NMDA antagonist if the patient's cellular DNA possesses a cytosine at said position.

In a further aspect of the invention there is provided a method of treating a patient who is a candidate for treatment with an NMDA antagonist comprising:
(i) determining whether the base of GABR-A2 in a sample obtained from the patient at the following position as defined in SEQ ID NO: 1: position 401, is cytosine; and
(ii) if the base determined in step (i) is cytosine, administering to said patient an effective amount of an NMDA antagonist.

In a further aspect of the invention there is provided a method of treating a patient who is a candidate for treatment with an NMDA antagonist comprising:
(i) determining whether the sequence of GABR-A2 in a sample obtained from the patient at the position 401 according to any of SEQ ID NOs: 1-4, is the minor allele; and
(ii) if the answer to step (i) is yes, administering to said patient an effective amount of an NMDA antagonist.
   In a further aspect of the invention there is provided a method of treatment comprising
(a) selecting a patient in need of treatment for depression or anxiety, the patient's genome having been identified as bearing a cytosine at single nucleotide polymorphism position rs3756007 in GABR-A2 gene; and
(b) treating the patient with an NMDA antagonist.

In a further aspect of the invention there is provided a method of making a marketable drug, the method comprising
(a) preparing a package containing an NMDA antagonist; and
   including in the package a label or printed inset recommending use of the NMDA antagonist for the treatment of depression or anxiety in a patient whose genome comprises a cytosine at single nucleotide polymorphism position rs3756007 in GABR-A2 gene, and/or a thymine at single nucleotide polymorphism position rs11503016 in GABR-A2 gene, and/or cytosine at single nucleotide polymorphism position rs17537359 in GABR-A2 gene, and/or a thymine at single nucleotide polymorphism position rs1372472 in GABR-A2 gene.

In a further aspect of the invention there is provided use of an NMDA antagonist in the manufacture of a medicament for the treatment of a patient identified as likely to be responsive to treatment with an NMDA antagonist according to the methods described above.

In a further aspect of the invention there is provided use of an NMDA antagonist in the manufacture of a medicament for the treatment of a patient with depression or anxiety whose cellular DNA has been determined to possess a cytosine at the position in the GABR-A2 gene corresponding to position 401 according to SEQ ID NO: 1, according to any of the methods described herein.

In a further aspect of the invention there is provided an NMDA antagonist for use in the treatment of a patient suffering from depression or anxiety, wherein the patient's cellular DNA has been determined to possess a cytosine in the GABR-A2 gene corresponding to position 401 (according to SEQ ID NO: 1).

In a further aspect of the invention there is provided an NMDA antagonist for use in the treatment of a patient suffering from depression or anxiety whose cellular DNA has been identified as possessing the minor allele at one or other of the rs3756007, rs11503016, rs17537359 or rs1372472 SNPs in GABR-A2 gene.

In a further aspect of the invention there is provided an NMDA antagonist drug for use in the treatment of depression or anxiety in one or more patients whose cellular DNA has previously been identified as possessing a cytosine at single nucleotide polymorphism position rs3756007, and/or a thymine at single nucleotide polymorphism position rs11503016, and/or cytosine at single nucleotide polymorphism position rs17537359, and/or a thymine at single nucleotide polymorphism position rs1372472, in GABR-A2 gene.

As noted above, the various aspects of the invention are suitable for use with patients suffering from major depressive disorder (MDD), patients with a single or recurrent depressive episodes, patients with treatment-refractory depression (i.e. patients who have been found to be non-responsive to other depression treatments; TRD), patients with bipolar depression, patients with general anxiety disorder (GAD), patients with obsessive compulsive disorder (OCD), patients with panic disorder, patients with post traumatic stress disorder (PTSD), and patients with social anxiety disorder.

As noted above, the following NMDA antagonist compounds: (S)-1-phenyl-2-(pyridin-2-yl)ethanamine (disclosed in WO 93/20052) and ketamine are particularly suitable for use in the various aspects of the invention.

### EXAMPLES

The invention is illustrated by the following non-limiting examples.

### Example 1

Genomic DNA samples were collected from individuals enrolled in a Phase 11 study of COMPOUND A (total number of patients in the study was 152). The Phase II study was designed to measure the efficacy of COMPOUND A in the treatment of depression. Patients were selected for enrolment in the aforementioned Phase II study on the basis of their prior treatment history which defined them as poor responders to medications used in the treatment of depression. Common genetic variations (single nucleotide polymorphisms, SNPs) in genes encoding brain derived neurotrophic factor (BDNF) and the alpha-2 subunit of the GABA_{A} receptor (GABR-A2) were tested using TaqMan® assays to measure their influence on the response to treatment for depression with COMPOUND A (an N-methyl-D-aspartate (NMDA) antagonist). Montgomery-Åsberg Depression Rating Scale (MADRS; Montgomery SA, Asberg M (April 1979). “A new depression scale designed to be sensitive to change”. British Journal of Psychiatry 134 (4): 382-89) was used as a measurement of treatment response. Thirty-two SNPs were selected for genotyping in GABR-A2 and six SNPs in BDNF.

Results of the genetic analysis of BDNF did not demonstrate a contribution to variation in treatment response. However, the results from the GABR-A2 gene did indicate an influence on change in MADRS from baseline for a number of SNPs, with the rs3756007 SNP showing the strongest association. Table 1 lists those genetic variations tested for which, on average, the minor allele positively influenced change in MADRS after treatment with COMPOUND A. The P value from the patients treated with the drug (treatment group) is also shown (therapeutic_pvalue).

TABLE 1

| | | | Additive | |
|---|---|---|---|---|
| | | | genetic | |
| | | | effect | |
| | | Minor allele | of minor | |
| SNP id | | and | allele on | |
| (Reference | Applied | frequency in | change in | Thera- |
| Sequence, | Biosystems ® | caucasian | *MADRS | peutic |
| rs, number) | Catalogue number | population | total score. | P value |
| rs11503016 | C_25637260_10 | T 0.134 | −5.33 | 0.0374 |
| rs17537359 | C_32680492_10 | C 0.099 | −9.40 | 0.0038 |
| rs3756007 | C_27496599_10 | C 0.073 | −12.84 | 0.0021 |
| rs1372472 | C_7537281_10 | T 0.338 | −4.83 | 0.0108 |

Thus, among the GABR-A2 SNPs tested, patients who were treated with COMPOUND A and carried the minor allele at rs3756007, rs11503016, rs17537359 and rs1372472, experienced, on average, greater improvement than carriers of the major allele homozygote.

### SNP Detection

TaqMan® assays were used to detect the the particular SNPs within the target genes. Each TaqMan® assay is specific for a given mutation or allele, i.e. designed to detect an alternate base relative to another base at a given position. each TaqMan® assay contains sequence-specific forward and reverse primers to amplify the polymorphic sequence of interest in the target gene along with TaqMan® probes labelled with different fluorescent tags to identify the target base (SNP). The TaqMan® assays were ordered from Applied Biosystems® and where possible Pre-Designed SNP Genotyping Assays were used. Each of the GABR-A2 SNPs showing association with an influence on the response to treatment for depression with COMPOUND A were detected using the commercially available Pre-Designed SNP Genotyping Assays. The catalogue number for each of the SNPs is listed in Table 1.

For those SNP where a Pre-Designed assay was not available, Custom TaqMan® SNP Genotyping Assays were used. Table 2 provides the primer and probe sequences and labels used for a representative custom designed SNP assay for rs490434.

For each SNP assay, genomic DNA was extracted from the clinical sample and the TaqMan® assay was carried out using the primers in Table 2. For the TaqMan® reaction approximately 10 ng of genomic DNA was used, in a total reaction volume of 2 ul. The reaction used TaqMan® Genotyping Master Mix (Applied Biosystems®, part number 4381657). The TaqMan® Genotyping Master Mix was diluted 1:1 with deionised water and the TaqMan® assay used at 80× concentration. The reaction was then securely sealed before Thermal Cycling. Cycle conditions: 95° C. for 10 minutes followed by 40 cycles of 92° C. for 15 seconds, 60° C. for 60 seconds in a Thermal Cycler instrument (KBiosystems DT-108 Thermal Cycler). The plate was then analysed on the Applied Biosystems® 7900HT Sequence Detection System using the pre-programmed “Allelic Discrimination” analysis in the Applied Biosystems® SDS v2.4 Software.

TABLE 2
rs490434 Oligonucleotide and
TaqMan ® Probe Sequences

| Primer | 5′ Mod | Sequence | 3′ Mod |
|---|---|---|---|
| TaqMan ® | | GTCATTTCCCAACCTCA | |
| Forward | | GGTCTT | |
| Primer | | (SEQ ID NO: 5) | |
| TaqMan ® | | TCACCTATTACATATAA | |
| Reverse | | GGCTTGTGAGGT | |
| Primer | | (SEQ ID NO: 6) | |
| TaqMan ® | FAM ™ | TTAATTTATTTCGCATT | Minor Groove |
| MGB | | TTT | Binder (MGB) |
| probes | | (SEQ ID NO: 7) | & |
| | | | nonfluorescent |
| | | | quencher |
| TaqMan ® | VIC ® | TGGTTAATTTATTTCAC | Minor Groove |
| MGB | | ATTTTT | Binder (MGB) |
| probes | | (SEQ ID NO: 8) | & |
| | | | nonfluorescent |
| | | | quencher |

1. A method for selecting a patient for treatment with an NMDA antagonist drug, comprising determining in a nucleic acid containing sample from said patient the nucleotide at single nucleotide polymorphism (SNP) position rs3756007 and/or, rs11503016, and/or rs17537359, and/or rs1372472 in GABR-A2 gene, wherein if there is a cytosine at rs3756007, or a thymine at rs11503016, or a thymine at rs11503016, or a thymine at rs1372472, said patient is selected for treatment with an NMDA antagonist drug.

2. A method of recommending a treatment, the method comprising
(a) selecting a patient in need of treatment for depression and/or anxiety, the patient's genome having been identified as bearing a minor allele at any one rs3756007, rs11503016, rs17537359 or rs1372472 in GABR-A2 gene; and
(b) recommending that the patient be treated with an NMDA antagonist.

3. The method as claimed in claim 1 or 2, wherein the NMDA antagonist drug is selected from the group consisting of: (S)-1-phenyl-2-(pyridin-2-yl)ethanamine and ketamine.

4. The method as claimed in claim 1 or 2, wherein the NMDA antagonist drug is (S)-1-phenyl-2-(pyridin-2-yl)ethanamine.

5. The method as claimed in any of the preceding claims wherein the depression and/or anxiety is selected from: major depressive disorder (MDD), single or recurrent depressive episodes, treatment-refractory depression (TRD), bipolar depression, general anxiety disorder (GAD), obsessive compulsive disorder (OCD), panic disorder, post traumatic stress disorder (PTSD), and social anxiety disorder.

6. The method as claimed in claim 1, wherein the nucleic acid containing sample is a solid tissue sample or a biofluid sample.

7. The method as claimed in any of the preceding claims, wherein the nucleotide at position rs3756007, rs11503016, rs17537359 or rs1372472 in GABR-A2 gene is determined by polymerase chain reaction (PCR), hybridization with allele specific probes or primers, allele specific amplification (such as amplification refractory mutation system—ARMS), enzymatic mutation detection, mass spectrometry, single strand conformation polymorphisms, restriction fragment length polymorphism (RFLP), WAVE analysis, denaturing gradient gel electrophoresis, high resolution melting or temperature gradient gel electrophoresis or nucleic acid sequencing.

8. The method as claimed in claim 7, wherein the nucleotide at position rs3756007, rs11503016, rs17537359 or rs1372472 in GABR-A2 gene is determined by sequencing or allele specific amplification.

9. Use of an oligonucleotide primer capable of determining the nucleotide at any one of rs3756007, rs11503016, rs17537359 or rs1372472 SNPs in GABR-A2 gene for predicting whether a patient suffering from depression is likely to respond favourably to treatment with an NMDA antagonist drug.

10. A method of treatment comprising
(a) selecting a patient in need of treatment for depression, the patient's genome having been identified as bearing a cytosine at single nucleotide polymorphism position rs3756007 in GABR-A2 gene; and
(b) treating the patient with an NMDA antagonist.

11. A method of treating a patient suffering from depression or anxiety comprising administering to a patient suffering from depression or anxiety whose cellular DNA has been determined to comprise the minor allele at any of rs3756007, rs11503016, rs17537359 or rs1372472 in GABR-A2 gene an effective amount of an NMDA antagonist drug.

12. A method of making a marketable drug, the method comprising
(a) preparing a package containing an NMDA antagonist; and
(b) including in the package a label or printed inset recommending use of the NMDA antagonist for the treatment of depression in a patient whose genome comprises a cytosine at single nucleotide polymorphism position rs3756007 in GABR-A2 gene.

13. The method as claimed in any of claims to 10-12, wherein the NMDA antagonist is selected from: (S)-1-phenyl-2-(pyridin-2-yl)ethanamine and ketamine.

14. An NMDA antagonist for use in the treatment of depression or anxiety in one or more patients whose cellular DNA has been determined to possesses a cytosine at the single nucleotide polymorphism known as rs3756007 in the GABR-A2 gene.

15. The NMDA antagonist as claimed in claim 14 which is selected from: (S)-1-phenyl-2-(pyridin-2-yl)ethanamine and ketamine.
